(19) United States
(12) Patent Application Publication
(10) Pub. No.: US 2007/0239091 A1
(43) Pub. Date: Oct. 11, 2007

(54) Shoulder stabilizing suspensory device with brace

(76) Inventors: William S. Brockington, Aiken, SC (US); Celeste Williams Brockington, Aiken, SC (US)

Correspondence Address: Poh C. Chua;PILLSBURY WINTHROP SHAW PITTMAN LLP, 1650 Tysons Boulevard, McLean, VA, 22102, US

(21) Appl. No.: 11/401,273

(22) Filed: Apr. 11, 2006

(51) Int. Cl. A61F 5/00

(52) U.S. Cl. 602/4

(57) ABSTRACT

A suspensory device for providing shoulder stabilization is disclosed having a chest strap, an underarm pad, and means for affixing the underarm pad to the suspensory device. According to another aspect, the suspensory device may have one or more shoulder straps. The suspensory device may also be adjustable both in the shoulder strap area as well as the chest strap. The suspensory device may also include underarm pads on both sides therefore allowing the suspensory device to be usable for either arm without repositioning of the suspensory device.

### BACKGROUND

1. Field of the Invention

The present invention relates generally to a device for maintaining proper position of the shoulder. More specifically, the present invention relates to a suspensory device, e.g., a sling, that houses a pad that fits under the arm to maintain proper position of the humeral head with respect to the glenoid.

2. Background of the Invention

There are two main bones in the shoulder: the humerus and the scapula, or shoulder blade. The joint cavity between these two bones is cushioned by articular cartilage covering the head of the humerus and the face of the glenoid. The glenoid fossa is positioned at the superiolateral aspect of the scapula. The head of the humerus sits in the glenoid fossa to form a ball and socket joint called the glenohumeral joint. At the superiolateral aspect of the scapula lies the glenoid fossa, which meets the humerus to form the glenohumeral joint. This joint is categorized as a flexible ball-and-socket joint. The labrum, a ring of fibrous cartilage surrounding the glenoid, serves to deepen and add stability to the glenohumeral joint.

Ligaments connect the bones of the shoulder and tendons join the bones to surrounding muscles. The biceps tendon attaches the biceps muscle to the shoulder and helps stabilize the joint. Four short muscles originate on the scapula and pass around the shoulder where their tendons fuse together to form the rotator cuff. All of these components of the shoulder, along with the muscles of the upper body, work together to manage the stress the shoulder receives as it is moved.

The deltoid is a three headed muscle that caps the shoulder. The three heads of the deltoid are the anterior, posterior, and lateral. All three deltoid heads attach to the humerus. The anterior and lateral heads originate on the clavicle, while the posterior head originates on the scapula. The anterior head raises it away to the front, the lateral head up and away to the side, and the posterior head away to the rear.

The supraspinatus tendon runs along the top of the scapula and inserts at the top of the arm. This muscle's major role is to stabilize the humeral head within the glenoid. It is also used to lift the arm up sideways. It is also important in throwing sports as it is the muscle that plays an important role in stabilizing the humerus within the glenoid fossa when whatever is being thrown is released. As may be expected, rupturing of the supraspinatus tendon can cause abnormal shoulder mechanics and can potentially cause further injury.

The pectoralis major muscle is a large powerful muscle at the front of the chest. It is used to rotate the arm inwards, pull a horizontal arm across the body, pull the arm from above the head down. This muscle is most likely to rupture at the point where it inserts into the arm, which can also affect arm/shoulder position.

A winged scapula is a condition in which the scapula or shoulder blade is abnormally rotated and sticks out at the back, particularly when pushing against something, such as a wall. The need for scapular stabilization is often forgotten and this can lead to just as big of a loss of function of the shoulder complex as a whole as a rotator cuff injury. If the scapula is not properly positioned there will be extra strain on the glenohumeral joint and the possibility of strain to the rotator cuff and the associated bursa as a secondary problem. The trapezius is often blamed for such problems as it is an essential muscle for scapular control, but the serratus anterior has been shown to be equally as important in the action of throwing, or other similar movements. Accordingly, muscular imbalance around the scapula can be just as much of a problem as imbalance between the rotator cuff and other shoulder muscles.

In any throwing motion (or any other heavy usage of the shoulder), the muscles, tendons, and bones work in an incredibly complex fashion. Unlike other joints that move in only two directions (e.g., the knee), the shoulder joint is freely movable to allow movements that accommodate a wide variety of motions. After heavy use, the muscles are often contracted rather than elastic. Inflammation of a joint is common from the friction. Inflammation can decrease the amount of room for the humerus to move freely on the glenoid fossa. Tendonitis is a common result. Accordingly, it is common to use ice to decrease pain and reduce inflammation. It would be desirable to have a device that assists in reducing stress on the glenohumeral joint, either after heavy usage or after a surgery, to maintain proper positioning of the joint thereby alleviating pain.

### BRIEF SUMMARY OF THE INVENTION

A suspensory device for providing shoulder stabilization is disclosed having a chest strap, an underarm pad, and means for affixing the underarm pad to the suspensory device.

According to another aspect of the invention, the suspensory device may have one or more shoulder straps.

### BRIEF DESCRIPTION OF THE DRAWINGS

FIG. 1 is a perspective view of a suspensory device according to an exemplary embodiment of the present invention.

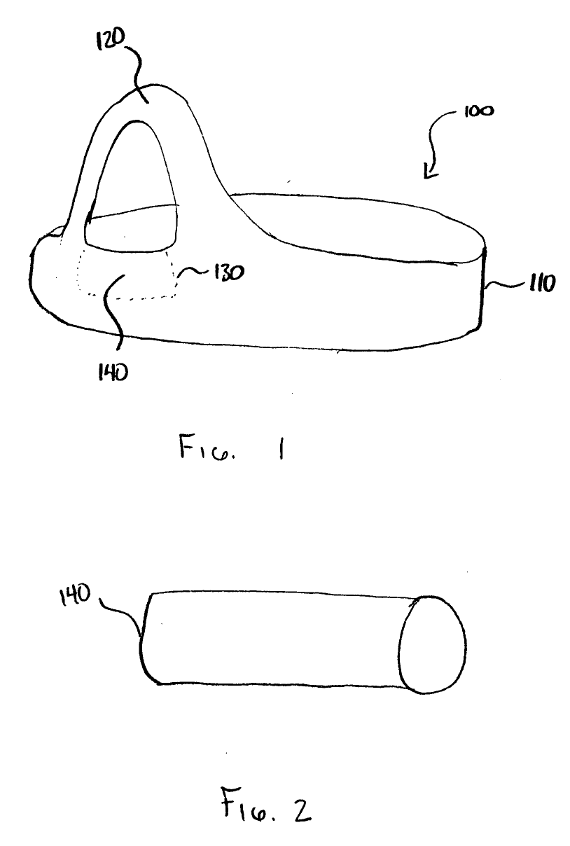

FIG. 2 is a perspective view of a pad for use with the suspensory device of FIG. 1.

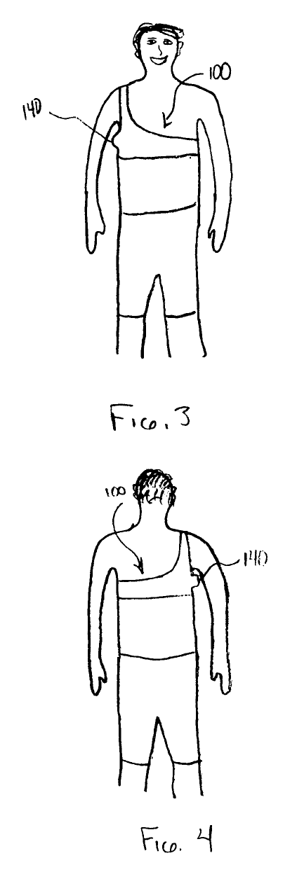

FIGS. 3-5 show the suspensory device of FIG. 1 in use by a person.

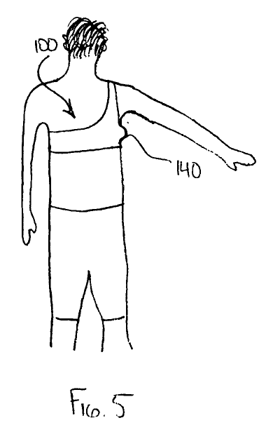

### DETAILED DESCRIPTION OF THE INVENTION

The present invention is a device that can be used to relieve stress on the shoulder. An exemplary embodiment of the invention is configured to be positioned under the shoulder joint, essentially in the armpit, to aid in proper positioning of the joint. Such a shoulder support accomplishes many favorable results. First, it distracts (pulls the humerus socket away from the glenoid) the humerus as well as pushes it away from the body and forces the labrum to expand. The net result reduces friction within the joint. Second, it reduces strain on the joint by shifting the weight of the arm, which weighs approximately 15-20 lbs. to the chest, thus bypassing the shoulder joint. This allows the soft tissues of the joint via the muscles, the capsule and the ligaments to relax thus decreasing the pain and improving the mobility of the shoulder with decreased stress in the joint.

In order to achieve such positioning it is known for physical therapists to suggest rolling up a sock or small towel and inserting it in the armpit. The present invention provides a more user-friendly improvement over the “rolled up sock” that holds the positioning device in place and maintains proper shoulder position without continued repositioning of the sock by the user.

The present invention essentially involves a type of a suspensory device intended to be worn by a user. As seen in Figure, sling 100, a preferred embodiment of the invention, includes a strap 110. Strap 110 preferably wraps around the chest of a user at approximately armpit level. Strap 110 could be a closed loop of elastic material to allow it to be easily taken on or off by the user. Strap 110 may also include, among other configurations, an open loop of material having a closure device to allow the suspensory device to be wrapped around the chest with the closure device being used to secure the strap in place around the user's chest.

Suspensory device 100 further includes at least one shoulder strap 120 that is positioned over the shoulder. Strap 120 is configured such that the suspensory device may be worn with a pad positioned in either the left or right armpit area. Strap 120 may also include and adjustment device as is common with many over-the-shoulder type clothing to allow for adjustment tailored to fit a range of users.

At the intersection of straps 110 and 120 is situated a pocket 130, or other such apparatus, for holding and positioning a removable spacer 140 in the armpit. Pocket 130 may simply be an open pocket of sufficient size to hold spacer 140 or it may also have a closure at the top or side to allow for the spacer to be held more securely within the pocket. This closure may be made of, for example, a hook and loop closure, snaps, zippers, or any other such suitable closure mechanism. There may be pockets 130 on only one side of strap 110. Alternatively, a pocket for spacer 140 may be located on both sides to allow for suspensory device 100 to be usable for either shoulder without repositioning of the suspensory device.

Spacer 140 replaces the rolled up sock and aids in maintaining proper shoulder position by forcing the humerus away from the glenoid, and thus reducing friction between the two. This proper positioning allows for a better environment for healing of the joint either after surgery or simply after heavy usage.

In an initial embodiment, spacer 140 consisted of the cylindrical hand-pad. An example of spacer 140 is the armpit support from a set of crutches. However, any pad that would sufficiently move the humerus away from the glenoid would suffice. Spacer 140 also is not limited to a cylindrical shape, but may be of any shape (e.g., square, rectangular, triangular, trapezoidal, etc.) that would provide proper positioning of the humerus with respect to the glenoid.

Suspensory device 100 also may include more than one shoulder strap 120. For example, suspensory device 100 may include two shoulder straps, each having a pocket or other spacer affixing means so that the device may be used on either or both shoulders. In another embodiment, suspensory device 100 may also have no shoulder straps at all, and include simply a chest strap with a pocket 130 for housing spacer 140. In such a strapless embodiment, suspensory device 100 may include a single pocket 130, or it could have pockets on either side to allow for use with either or both shoulders.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A suspensory device for providing shoulder stabilization, comprising:
a chest strap;
an underarm pad; and
means for affixing the underarm pad to the suspensory device.

2. The suspensory device of claim 1 wherein the underarm pad is positioned such that when worn by a user, the pad forces top of the humerus bone away from the glenoid.

3. The suspensory device of claim 1, wherein the pad has a cylindrical-shape.

4. The suspensory device of claim 1, wherein the pad is padded to provide comfort to its user.

5. The suspensory device of claim 1, wherein the pad comprises a soft rubber material.

6. The suspensory device of claim 1, wherein the pad comprises a foam material.

7. The suspensory device of claim 1, further comprising at least one shoulder strap.

8. The suspensory device of claim 1, further comprising two shoulder straps.

9. The suspensory device of claim 1, wherein the chest strap comprises an elastic material.

10. The suspensory device of claim 1, wherein the chest strap includes a closure device.

11. The suspensory device of claim 1, wherein the means for affixing comprises a pocket formed in or affixed to the chest strap.

12. The suspensory device of claim 11, wherein the pocket comprises a pocket closure device.

13. The suspensory device of claim 1, wherein the chest strap is adjustable.

14. The suspensory device of claim 7, wherein the at least one shoulder strap is adjustable.

15. The suspensory device of claim 1, wherein the pad comprises a cylindrically shaped pad.

16. A method for providing shoulder stabilization, comprising:
selecting an underarm pad; and
affixing the underarm pad to a chest strap, whereby the underarm pad positions a shoulder joint of a user to relieve stress when the chest strap is worn around the chest of the user.

17. The method of claim 16, further comprising:
attaching at least one shoulder strap to the chest strap, the shoulder strap is configured to rest over the user's shoulder.

18. A suspensory device for providing shoulder stabilization, comprising:
an underarm pad, wherein the underarm pad is configured to be rigid;
a chest strap configured to house the underarm pad, wherein the chest strap is configured to be worn by a user such that the underarm pad is positioned under an armpit area of the user.

19. The suspensory device of claim 18, wherein the chest strap comprises a pocket that houses the underarm pad.

20. The suspensory device of claim 18, further comprising attaching a shoulder strap to the chest strap, the shoulder strap is configured to rest over a shoulder of the user.
